(19) United States
(12) Patent Application Publication
(10) Pub. No.: US 2021/0072246 A1
(43) Pub. Date: Mar. 11, 2021

(54) BIOMARKERS FOR DETECTION OF OVARIAN CANCER

(71) Applicant: PROVISTA DIAGNOSTICS, INC., Alpharetta, GA (US)

(72) Inventors: Meredith C. Henderson, Mesa, AZ (US); Rao Mulpuri, Apex, NC (US); David E. Reese, New York, NY (US)

(21) Appl. No.: 16/895,943

(22) Filed: Jun. 8, 2020

Related U.S. Application Data
(63) Continuation of application No. 15/087,456, filed on Mar. 31, 2016, now abandoned.
(60) Provisional application No. 62/142,316, filed on Apr. 2, 2015.

(51) Int. Cl. G01N 33/574

(52) U.S. Cl. CPC G01N 33/57449

(57) ABSTRACT

Methods are provided for predicting and diagnosing the presence of ovarian cancer, as well as for assessing the therapeutic efficacy of a cancer treatment and determining whether a subject potentially is developing cancer.

### CROSS REFERENCE TO RELATED APPLICATION(S)

The present application is a continuation application of U.S. patent application Ser. No. 15/087,456, filed on Mar. 31, 2016, now pending, which claims the benefit under 35 USC § 119(e) of U.S. Application Ser. No. 62/142,316, filed Apr. 2, 2015, now expired. The entirety of the aforementioned applications is incorporated herein by reference.

### BACKGROUND

### Field of Invention

The present invention relates generally to methods for cancer detection, and more particularly to methods for predicting and diagnosing ovarian cancer.

### Background Information

The American Cancer Society estimates that ovarian cancer will strike 22,430 women and take the lives of 15,280 women in 2007 in the United States. Ovarian cancer is not a single disease, however, and there are actually more than 30 types and subtypes of ovarian malignancies, each with its own pathology and clinical behavior. Most experts therefore group ovarian cancers within three major categories, according to the kind of cells from which they were formed: epithelial tumors arise from cells that line or cover the ovaries; germ cell tumors originate from cells that are destined to form eggs within the ovaries; and sex cord-stromal cell tumors begin in the connective cells that hold the ovaries together and produce female hormones.

Ovarian cancer is by far the most deadly of gynecologic cancers, accounting for more than 55 percent of all gynecologic cancer deaths. But ovarian cancer is also among the most treatable—if it is caught early. When ovarian cancer is caught early and appropriately treated, the 5-year survival rate is 93 percent.

The current reality for the diagnosis of ovarian cancer is that most cases (81 percent of all cases of ovarian cancer) are not caught in earliest stage. This is because early stage ovarian cancer is very difficult to diagnose. Its symptoms may not appear or be noticed during the very early initial stages of cancer development. If symptoms are present (e.g. bloating, indigestion, diarrhea, constipation and others), they are often vague and associated with many common and less serious conditions. Most importantly, there has been no effective test for early detection. An effective tool for early and accurate detection of ovarian cancer is a critical unmet medical need.

In this setting, there is intense effort in the search for biomarkers that can detect early disease and/or monitor for disease progression and recurrence. With the advent of molecularly-targeted therapeutics, biomarkers that are associated with biological subtypes of cancer may be useful for predicting responses to therapeutic interventions.

Protein-based approaches to distinguish cancer-bearing patient sera from healthy control sera have been challenged by the difficulty in identifying small quantities of protein fragments within complex protein mixtures, protein instability, and natural variations in protein content within patient populations. Autoantibodies (AAb) to tumor antigens have advantages as potential cancer biomarkers as they are stable, highly specific, easily purified from serum, and are readily detected with well-validated secondary reagents. Although they have high specificities to distinguish cancer from control sera, most tumor-associated autoantibodies (TAAbs) demonstrate poor sensitivities. Testing multiple antigens in parallel may serve to increase the predictive value of tumor-specific antibodies for use as immunodiagnostics.

There are several platforms that may be utilized to screen for immune responses using tumor antigens. For example, protein microarrays offer a platform to present tumor antigens to screen for immune responses. Protein microarrays are capable of presenting and assessing hundreds of tumor antigens simultaneously. The responses are rapidly identified because the position of each protein is known in advance and there are no representation issues; all proteins, even rare ones, are represented equally (usually in duplicate). The proteins are arrayed on a single microscope slide requiring only a few microliters of serum per assay. Known tumor antigens as well as predicted tumor antigens can be included to generate a comprehensive protein tumor antigen array.

What has been urgently needed in the field of gynecologic oncology is a minimally invasive clinical test for assessing and predicting the presence of ovarian cancer that is based on a robust set of biomarkers identified from a large and diverse set of samples.

### SUMMARY OF THE INVENTION

The present invention generally relates to cancer biomarkers and particularly to biomarkers associated with ovarian cancer. It provides methods to predict, evaluate, diagnose, and monitor cancer, particularly ovarian cancer, by measuring certain biomarkers. A set of biomarkers including serum protein biomarkers and TAAbs provides a detectable molecular signature of ovarian cancer in a subject.

Accordingly, in one embodiment, the invention provides a method for determining whether a subject has or is at risk of having ovarian cancer. The method includes obtaining a biological sample from the subject and measuring a level of at least one autoantibody in the sample and at least one protein biomarker, both as compared with a healthy subject's sample (e.g., a corresponding normal sample not having ovarian cancer); wherein a level of antibody and biomarker greater than that found in the healthy sample, is indicative of a subject having or at risk of having ovarian cancer.

In another embodiment, the method includes: a) obtaining a biological sample from the subject; b) measuring a level of at least one protein biomarker and at least one autoantibody; c) determining whether the level is elevated; and d) providing a determination of whether the subject has or is at risk of having ovarian cancer.

In another embodiment, the invention provides a method for measuring the level of a protein biomarker and an autoantibody in a sample from a subject having or at risk of having ovarian cancer. The method includes: a) obtaining a biological sample from the subject; and b) measuring a level of at least one protein biomarker and at least one autoantibody, In embodiments, the at least one protein biomarker is selected from CA125, CA15.3, CA19-9, HE4, Prolactin, APOA1, CLDN1, CLDN3, CLDN4, CLDN5, CLDN7, VEGF, LPA, AFP, LH, CEA, FSH, and any combination thereof, and the at least one autoantibody specifically binds RAB7L1, ACSBG1, AFP, CSNK1A1L, DHFR, MBNL1, p53, PRL, PSMC1, PTGFR, PTPRA, SCYL3, or any combination thereof.

In embodiments, the level of the at least one protein biomarker is determined by protein array analysis.

In yet another embodiment, the present invention provides a kit for detecting ovarian cancer in a subject. The kit includes means for detecting in a biological sample at least one protein biomarker and at least one autoantibody.

In another embodiment, the present invention provides an array comprising a plurality of probes for specifically binding a biomarker or autoantibody. The probes may include oligonucleotides or polypeptides.

A panel of biomarkers for use with the invention includes the following proteins or fragments thereof: CA125, CA15.3, CA19-9, HE4, Prolactin, APOA1, CLDN1, CLDN3, CLDN4, CLDN5, CLDN7, VEGF, LPA, AFP, LH, CEA and FSH.

A panel of biomarkers for use with the invention includes the following proteins or fragments thereof: CA125, CA15.3, CA19-9, HE4, Prolactin, LPA, AFP, LH, CEA and FSH.

A panel of biomarkers for use with the invention includes one or more TAAbs which specifically bind RAB7L1, ACSBG1, AFP, CSNK1A1L, DHFR, MBNL1, p53, PRL, PSMC1, PTGFR, PTPRA, SCYL3, or a combination thereof, as well as variants (including partial or mutant) thereof.

A panel of biomarkers for use with the invention includes one or more of the following proteins or fragments thereof: CA125, CA15.3, CA19-9, HE4, Prolactin, APOA1, CLDN1, CLDN3, CLDN4, CLDN5, CLDN7, VEGF, LPA, AFP, LH, CEA and FSH; in combination with autoantibodies that specifically bind one or more of RAB7L1, ACSBG1, AFP, CSNK1A1L, DHFR, MBNL1, p53, PRL, PSMC1, PTGFR, PTPRA, and SCYL3.

A panel of biomarkers for use with the invention includes one or more of the following proteins or fragments thereof: CA125, CA15.3, CA19-9, HE4, Prolactin, LPA, AFP, LH, CEA and FSH; in combination with autoantibodies that specifically bind one or more of RAB7L1, ACSBG1, AFP, CSNK1A1L, DHFR, MBNL1, p53, PRL, PSMC1, PTGFR, PTPRA, and SCYL3.

### BRIEF DESCRIPTION OF THE DRAWINGS

FIG. 1 is a series of graphical representations presenting experimental data of the concentration of individual serum protein biomarkers detected in patient samples in one embodiment of the invention. Serum was analyzed using the Abbott Architect® platform. No evidence of ovarian disease (ND) (n=22) and ovarian cancer (OvCa) Samples (n=59).

FIG. 2 is a series of graphical representations presenting experimental data of the concentration of individual serum protein biomarkers detected in patient samples in one embodiment of the invention. Serum was analyzed using the Abbott Architect Plus® platform. No evidence of ovarian disease (ND) (n=22) and ovarian cancer (OvCa) Samples (n=59).

FIG. 3 is a graphical representation presenting experimental data of plasma LPA levels in patient samples in one embodiment of the invention. Levels were measured by ELISA. ND (n=24) and OvCa Samples (n=25).

FIG. 4 is a graphical representation presenting experimental data of LPA levels in serum and matched plasma in healthy individuals (n=22) in one embodiment of the invention. Levels were measured by ELISA.

FIG. 5 is a tabular representation showing the number and source of samples screened using the methodology of the disclosure in embodiments of the invention.

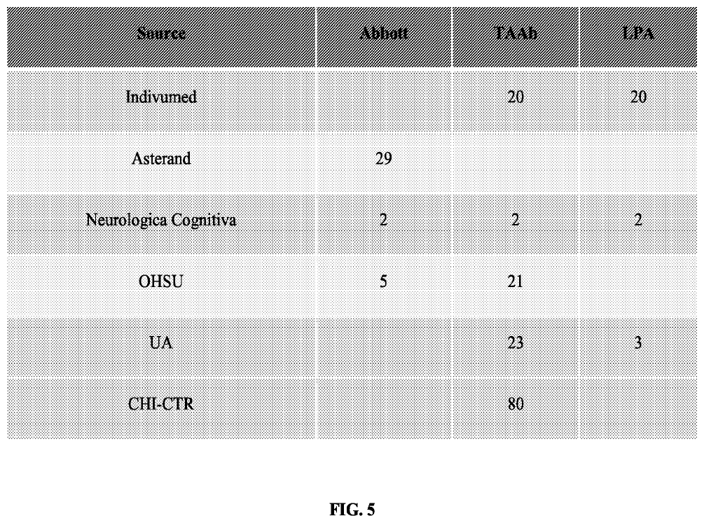

FIG. 6 is a graphical representation presenting experimental results of biomarker evaluation in one embodiment of the invention. The graph depicts non-weighted aggregate biomarker evaluation of n=35 ovarian cancer patients and n=17 healthy women. All ovarian cancer serum samples were collected post-biopsy. Biomarker concentration values are shown according to the legend for each sample.

FIG. 7 is a graphical representation presenting experimental data of TAAb detection in patient samples in one embodiment of the invention. A total of 12 TAAb targets (RAB7L1, ACSBG1, AFP, CSNK1A1L, DHFR, MBNL1, p53, PRL, PSMC1, PTGFR, PTPRA and SCYL3) were screened by indirect ELISA. Benign gynecological disease (BGD) (n=63) and OvCa Samples (n=111).

FIG. 8 is a graphical representation presenting experimental proof-of-concept model development using TAAbs (RAB7L1, ACSBG1, AFP, CSNK1A1L, DHFR, MBNL1, p53, PRL, PSMC1, PTGFR, PTPRA and SCYL3) to differentiate BGD (n=63) and OvCa samples (n=111). Sensitivity and specificity for this model were 70.3% and 71.4%, respectively. Positive-predictive value (PPV) for this model is 81.3%.

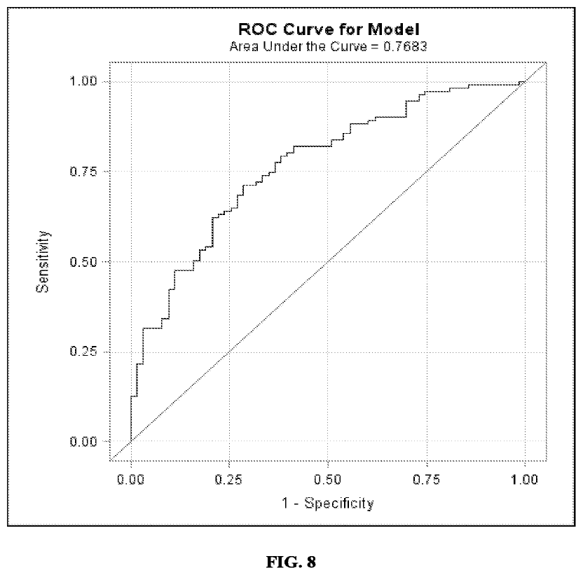

### DETAILED DESCRIPTION OF THE INVENTION

The present invention relates to biomarkers associated with ovarian cancer. It provides methods to predict, evaluate, diagnose, and monitor cancer, particularly ovarian cancer, by measuring certain biomarkers. A set of biomarkers including serum protein biomarkers and TAAbs provides a detectable molecular signature of ovarian cancer in a subject.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The presently disclosed subject matter provides a panel of biomarkers including proteins, specifically serum proteins, in combination with TAAbs, that are useful for the detection, desirably early detection, of ovarian cancer. The panel of biomarkers provided herein addresses certain limitations of early detection of tumors by other methods of screening alone.

Several proteins were assessed. In various embodiments, the panel includes one or more of the following proteins as well as fragments thereof: CA125, CA15.3, CA19-9, HE4, Prolactin, APOA1, CLDN1, CLDN3, CLDN4, CLDN5, CLDN7, VEGF, LPA, AFP, LH, CEA and FSH. In one embodiment, the panel includes CA-125, CA15.3, CA19-9, HE4 and Prolactin. In another embodiment, the panel includes CA125, CA15.3, CA19-9, HE4, Prolactin, LPA, AFP, LH, CEA and FSH

In combination with protein detection, the presently disclosed methodology utilizes detection of TAAbs, such as one or more TAAbs, each TAAB being specific for RAB7L1, ACSBG1, AFP, CSNK1A1L, DHFR, MBNL1, p53, PRL, PSMC1, PTGFR, PTPRA, or SCYL3. Additionally, multiple TAAbs may be utilized, wherein each of the multiple TAABs is specific for only one of RAB7L1, ACSBG1, AFP, CSNK1A1L, DHFR, MBNL1, p53, PRL, PSMC1, PTGFR, PTPRA, or SCYL3.

In various embodiments detection of TAAbs may be performed using any isoform or variant of RAB7L1, ACSBG1, AFP, CSNK1A1L, DHFR, MBNL1, p53, PRL, PSMC1, PTGFR, PTPRA, and SCYL3, including wild-type, mutant, as well as protein fragments thereof.

In combination, the presently disclosed biomarkers provide significant clinical utility for the early detection of ovarian cancer. Accordingly, in some embodiments methods are provided for assigning a subject to a group having a higher or lower probability of ovarian cancer. In one embodiment, the method includes determining the level of each of a panel of biomarkers in a sample from the patient, wherein the panel comprises at least one protein, or fragment thereof, selected from CA125, CA15.3, CA19-9, HE4, Prolactin, APOA1, CLDN1, CLDN3, CLDN4, CLDN5, CLDN7, VEGF, LPA, AFP, LH, CEA and FSH, and at least one TAAb selected from TAAbs specific for RAB7L1, ACSBG1, AFP, CSNK1A1L, DHFR, MBNL1, p53, PRL, PSMC1, PTGFR, PTPRA, and SCYL3, and assigning the patient to the group having a higher or lower probability of ovarian cancer based on the determined amount or relative presence/absence of each biomarker in the panel.

In some embodiments, a method is provided for assigning a subject to a high-risk group for ovarian cancer.

In some embodiments, a method is provided for managing treatment of a subject with potential ovarian cancer.

In various embodiments, the method of the present invention provides a sensitivity/specificity greater than use of SPBs or TAAbs alone. For example, the method of the present invention provides a sensitivity/specificity of detection greater than about 80, 81, 82, 83, 84, 85, 86, 87, 88, 89, 90, 91, 92, 93, 94, 95, 96, 97, 98 or 99% utilizing SPBs in combination with AAbs. In one embodiment, the method of the present invention provides a sensitivity/specificity of detection greater than about 80% utilizing SPBs in combination with AAbs.

The level of each of the presently disclosed panel of biomarkers can be determined in a variety of tissues. In some embodiments, the biomarkers can be detected in samples from a subject, which include bodily fluids such as, but not limited to, serum, blood, blood plasma, urine, sputum, seminal fluid, cerebrospinal fluid, ascites, feces, lymph or nipple aspirate, and the like.

In some embodiments, the presently disclosed methods can comprise statistically analyzing the amounts of each biomarker. The statistical analysis can comprise applying a predetermined algorithm to the amounts of the biomarkers. The results of the algorithm can be employed to assign a subject to a group having a higher or lower likelihood of ovarian cancer.

A “biomarker” in the context of the present invention is a molecular indicator of a specific biological property; a biochemical feature or facet that can be used to measure the progress of disease or the effects of treatment. “Biomarker” encompasses, without limitation, serum proteins and TAAbs, including their polymorphisms, mutations, variants, modifications, subunits, fragments, complexes, unique epitopes, and degradation products.

The term “polypeptide” is used in its broadest sense to refer to a polymer of subunit amino acids, amino acid analogs, or peptidomimetics, including proteins and peptoids. The polypeptides may be naturally occurring full length proteins or fragments thereof, processed forms of naturally occurring polypeptides (such as by enzymatic digestion), chemically synthesized polypeptides, or recombinantly expressed polypeptides. The polypeptides may comprise D- and/or L-amino acids, as well as any other synthetic amino acid subunit, and may contain any other type of suitable modification, including but not limited to peptidomimetic bonds and reduced peptide bonds.

In one embodiment, the disclosed methodology utilizes detection of TAAbs. As such, the method may utilize detection of various antibodies that bind different “antigenic fragments” or variants or mutant proteins. In one embodiment, the disclosed methodology utilizes detection of one or more RAB7L1 TAAb, one or more ACSBG1 TAAb, one or more AFP TAAb, one or more CSNK1A1L TAAb, DHFR TAAb, one or more MBNL1 TAAb, one or more p53 TAAb, one or more PRL TAAb, one or more PSMC1 TAAb, one or more PTGFR TAAb, one or more PTPRA TAAb, or one or more SCYL3 TAAb. As such, the method may utilize detection of various antibodies that bind different “antigenic fragments” of RAB7L1, ACSBG1, AFP, CSNK1A1L, DHFR, MBNL1, p53, PRL, PSMC1, PTGFR, PTPRA or SCYL3. As used herein, an “antigenic fragment” is any portion of at least 4 amino acids of a polypeptide that can give rise to an immune response. In various embodiments, an antigenic fragment is between 5-300 amino acids, or the full amino acid sequence of a given polypeptide. In various embodiments, an antigenic fragment is at least 5, 6, 7, 8, 9, 10, 11, 12, 13, 14, 151, 16, 17, 18, 19, 20, 21, 22, 23, 24, 25, 26, 27, 28, 29, 30, 35, 40, 45, 50, 75, 100, 150, 200, 250, 300, or the full amino acid sequence of a given polypeptide.

A variety of algorithms can be employed in the presently disclosed methods. The algorithms employed are not limited to those described herein, but rather include algorithms as would be apparent to those of ordinary skill in the art upon a review of the instant disclosure.

The level of each of a panel of biomarkers can be determined in the presently disclosed method. In some embodiments, the panel of biomarkers can comprise one or more serum proteins and at least one or more TAAbs. However, the presently disclosed subject matter is not limited to the panel of biomarkers described above. Any marker that correlates with ovarian cancer or the progression of ovarian cancer can be included in the biomarker panel provided herein, and is within the scope of the presently disclosed subject matter. Any suitable method can be utilized to identify additional ovarian cancer biomarkers suitable for use in the presently disclosed methods. For example, biomarkers that are known or identified as being up or down-regulated in ovarian cancer using methods known to those of ordinary skill in the art can be employed. Additional biomarkers can include one or more of polypeptides, small molecule metabolites, lipids, and nucleotide sequences. Markers for inclusion on a panel can be selected by screening for their predictive value using any suitable method including, but not limited to, those described.

As is apparent from the foregoing embodiments, the presently disclosed method is useful for screening patients for ovarian cancer, for the early detection of ovarian cancer, and for managing the treatment of patients with potential ovarian cancer or with known ovarian cancer. For example, in some embodiments, the panel of biomarkers can be useful for screening patients prior to imaging or other known methods for detecting ovarian tumors, to define patients at high risk or higher risk for ovarian cancer. Further, the presently disclosed method may be utilized in combination with other screening methods, such as imaging or histological analysis.

In one embodiment, the presence of any amount of biomarker in a sample from a subject at risk of ovarian cancer can indicate a likelihood of ovarian cancer in the subject. In another embodiment, if biomarkers are present in a sample from a subject at risk of ovarian cancer, at levels which are higher than that of a control sample (a sample from a subject who does not have ovarian cancer) than the subject at risk of ovarian cancer has a likelihood of ovarian cancer. Subjects with a likelihood of ovarian cancer can then be tested for the actual presence of ovarian cancer using standard diagnostic techniques known to the skilled artisan, including biopsy, histological analysis or imaging, such as MRI. In various embodiments, the method results in an accurate diagnosis in at least 70% of cases; more preferably of at least 75%, 80%, 85%, 90%, or more of the cases.

Any suitable method can be employed for determining the level of each of the panel of biomarkers, as would be apparent to one skilled in the art upon a review of the present disclosure. For example, a method for detecting TAAbs may include use of biomolecules immobilized on a solid support or substrate. In one embodiment, standard plate-based indirect ELISA assays can be used. Indirect ELISAs are run by coating blank ELISA plates with the target proteins encompassed within the panel. Human serum is added to the plate and target-specific TAAbs are detected with specific secondary reagents. Other suitable immobilization methods include, but are not limited to luciferase immunoprecipitation systems (LIPS), Luminex™ beads, mass spectrophotometer, standard immune dipstick assays, Nucleic Acid Protein Programmable Array (NAPPA) technology, and microbead-based ELISA assays.

As used herein, an array may be any arrangement or disposition of the polypeptides. In one embodiment, the polypeptides are at specific and identifiable locations on the array. Those of skill in the art will recognize that many such permutations of the polypeptides on the array are possible. In another non-limiting embodiment, each distinct location on the array comprises a distinct polypeptide.

Any suitable support or surface may be used. Examples of such supports include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g. gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots).

In one embodiment, the support is a solid support. Any suitable “solid support” may be used to which the polypeptides can be attached including but not limited to dextrans, hydrogels, silicon, quartz, other piezoelectric materials such as langasite, nitrocellulose, nylon, glass, diazotized mem branes (paper or nylon), polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

A variety of detection techniques are also suitable for detection of serum proteins. For example, methods for detecting proteins can include gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS), and matrix-assisted laser desorption/ionization time-of-flight mass spectrometry (MALDI-TOF MS).

In some embodiments, protein biomarkers can be detected using technologies well known to those of skill in the art such as gel electrophoresis, immunohistochemistry, and antibody binding. Methods for generating antibodies against a polypeptide of interest are well known to those of ordinary skill in the art. An antibody against a protein biomarker of the presently disclosed subject matter can be any monoclonal or polyclonal antibody, so long as it suitably recognizes the protein biomarker. In some embodiments, antibodies are produced using the protein biomarker as the immunogen according to any conventional antibody or antiserum preparation process. The presently disclosed subject matter provides for the use of both monoclonal and polyclonal antibodies. In addition, a protein used herein as the immunogen is not limited to any particular type of immunogen. For example, fragments of the protein biomarkers of the presently disclosed subject matter can be used as immunogens. The fragments can be obtained by any method including, but not limited to, expressing a fragment of the gene encoding the protein, enzymatic processing of the protein, chemical synthesis, and the like.

Antibodies of the presently disclosed subject matter can be useful for detecting the protein biomarkers. For example, antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, and the like), complement fixation assays, immunofluorescence assays, protein A assays, and immunoelectrophoresis assays, and the like. Upon review of the present disclosure, those skilled in the art will be familiar with numerous specific immunoassay formats and variations thereof that can be useful for carrying out the methods of the presently disclosed subject matter.

In any embodiment of the invention, detection techniques may utilize a detectable tag, such as a detectable moiety. A tag may be linked to a polypeptide through covalent bonding, including, but not limited to, disulfide bonding, hydrogen bonding, electrostatic bonding, recombinant fusion, and conformational bonding. Alternatively, a tag may be linked to a polypeptide by means of one or more linking compounds. Techniques for conjugating tags to polypeptides are well known to the skilled artisan. Detectable tags can be used diagnostically to, for example, assess the presence of antibodies, or antibodies to a protein in a sample; and thereby detect the presence of ovarian cancer, or monitor the development or progression of ovarian cancer as part of a clinical testing procedure. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, and the like. For immunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes.

Gene expression levels may be determined in a disclosed method using any technique known in the art. Exemplary techniques include, for example, methods based on hybridization analysis of polynucleotides (e.g., genomic nucleic acid sequences and/or transcripts (e.g., mRNA)), methods based on sequencing of polynucleotides, methods based on detecting proteins (e.g., immunohistochemistry and proteomics-based methods).

The assays described herein can be adapted to be performed by lay users without a laboratory. The users may be health care professionals in point-of-care facilities or lay consumers in field conditions. The devices may have multiple embodiments including single-use devices, simple reusable devices and computerized biomonitors. The single-use devices, similar to over-the-counter lateral flow assays for pregnancy, enable subjective multi-biomarker assays to be performed. Simple reusable devices also enable objective biomarker assays that provide a refined or enhanced indication of solid state cancer mass, and may also enable remote data processing.

Gene expression levels also can be determined by quantification of a microRNA or gene transcript (e.g., mRNA). Commonly used methods known in the art for the quantification of mRNA expression in a sample include, without limitation, northern blotting and in situ hybridization; RNAse protection assays; and PCR-based methods, such as reverse transcription polymerase chain reaction (RT-PCR) and real time quantitative PCR (also referred to as qRT-PCR). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE), and gene expression analysis by massively parallel signature sequencing (MPSS).

Some method embodiments involving the determination of mRNA levels utilize RNA (e.g., total RNA) isolated from a target sample, such as an ovarian cancer tissue sample. General methods for RNA (e.g., total RNA) isolation are well known in the art and are disclosed in standard textbooks of molecular biology.

Differential gene expression also can be determined using microarray techniques. In these methods, specific binding partners, such as probes (including cDNAs or oligonucleotides) specific for RNAs of interest or antibodies specific for proteins of interest are plated, or arrayed, on a microchip substrate. The microarray is contacted with a sample containing one or more targets (e.g., microRNA, mRNA or protein) for one or more of the specific binding partners on the microarray. The arrayed specific binding partners form specific detectable interactions (e.g., hybridized or specifically bind to) their cognate targets in the sample of interest.

In some examples, differential gene expression is determined using in situ hybridization techniques, such as fluorescence in situ hybridization (FISH) or chromogen in situ hybridization (CISH). In these methods, specific binding partners, such as probes labeled with a fluorophore or chromogen specific for a target cDNA, microRNA or mRNA (e.g., a biomarker cDNA or mRNA molecule or microRNA molecule) is contacted with a sample, such as an ovarian cancer sample mounted on a substrate (e.g., glass slide). The specific binding partners form specific detectable interactions (e.g., hybridized to) their cognate targets in the sample. For example, hybridization between the probes and the target nucleic acid can be detected, for example by detecting a label associated with the probe. In some examples, microscopy, such as fluorescence microscopy, is used.

In various embodiments, biomarkers of the present invention are differentially expressed as compared to a corresponding healthy or normal sample. For example one or more of CA125, CA15.3, CA19-9, HE4, Prolactin, APOA1, CLDN1, CLDN3, CLDN4, CLDN5, CLDN7, VEGF, LPA, AFP, LH, CEA and FSH may be differentially expressed. In one embodiment, CA125, CA15.3, CA19-9, HE4, Prolactin, APOA1, CLDN1, CLDN3, CLDN4, CLDN5, CLDN7, VEGF, LPA, AFP, LH, CEA and FSH expression is increased as compared to a corresponding healthy sample. In another embodiment, CA125, CA15.3, CA19-9, HE4, Prolactin, APOA1, CLDN1, CLDN3, CLDN4, CLDN5, CLDN7, VEGF, LPA, AFP, LH, CEA and FSH expression is decreased as compared to a corresponding healthy sample.

In an embodiment, CA125 expression is increased as compared to a corresponding healthy sample. In another embodiment, CA125 expression is decreased as compared to a corresponding healthy sample.

In an embodiment, CA15.3 expression is increased as compared to a corresponding healthy sample. In another embodiment, CA15.3 expression is decreased as compared to a corresponding healthy sample.

In an embodiment, CA19-9 expression is increased as compared to a corresponding healthy sample. In another embodiment, CA19-9 expression is decreased as compared to a corresponding healthy sample.

In an embodiment, HE4 expression is increased as compared to a corresponding healthy sample. In another embodiment, HE4 expression is decreased as compared to a corresponding healthy sample.

In an embodiment, Prolactin expression is increased as compared to a corresponding healthy sample. In another embodiment, Prolactin expression is decreased as compared to a corresponding healthy sample.

In an embodiment, APOA1 expression is increased as compared to a corresponding healthy sample. In another embodiment, APOA1 expression is decreased as compared to a corresponding healthy sample.

In an embodiment, CLDN1 expression is increased as compared to a corresponding healthy sample. In another embodiment, CLDN1 expression is decreased as compared to a corresponding healthy sample.

In an embodiment, CLDN3 expression is increased as compared to a corresponding healthy sample. In another embodiment, CLDN3 expression is decreased as compared to a corresponding healthy sample.

In an embodiment, CLDN4 expression is increased as compared to a corresponding healthy sample. In another embodiment, CLDN4 expression is decreased as compared to a corresponding healthy sample.

In an embodiment, CLDN5 expression is increased as compared to a corresponding healthy sample. In another embodiment, CLDN5 expression is decreased as compared to a corresponding healthy sample.

In an embodiment, CLDN7 expression is increased as compared to a corresponding healthy sample. In another embodiment, CLDN7 expression is decreased as compared to a corresponding healthy sample.

In an embodiment, VEGF expression is increased as compared to a corresponding healthy sample. In another embodiment, VEGF expression is decreased as compared to a corresponding healthy sample.

In an embodiment, LPA expression is increased as compared to a corresponding healthy sample. In another embodiment, LPA expression is decreased as compared to a corresponding healthy sample.

In an embodiment, AFP expression is increased as compared to a corresponding healthy sample. In another embodiment, AFP expression is decreased as compared to a corresponding healthy sample.

In an embodiment, LH expression is increased as compared to a corresponding healthy sample. In another embodiment, LH expression is decreased as compared to a corresponding healthy sample.

In an embodiment, CEA expression is increased as compared to a corresponding healthy sample. In another embodiment, CEA expression is decreased as compared to a corresponding healthy sample.

In an embodiment, FSH expression is increased as compared to a corresponding healthy sample. In another embodiment, FSH expression is decreased as compared to a corresponding healthy sample.

In various embodiments, the level of TAAbs of the present invention is increased as compared to a corresponding healthy sample. In various embodiments, the level of TAAbs of the present invention is decreased as compared to a corresponding healthy sample. In one embodiment, the level of TAAbs which specifically bind RAB7L1, ACSBG1, AFP, CSNK1A1L, DHFR, MBNL1, p53, PRL, PSMC1, PTGFR, PTPRA and SCYL3 is increased as compared to a corresponding healthy sample. In one embodiment, the level of TAAbs which specifically bind RAB7L1, ACSBG1, AFP, CSNK1A1L, DHFR, MBNL1, p53, PRL, PSMC1, PTGFR, PTPRA and SCYL3 is decreased as compared to a corresponding healthy sample.

In one embodiment, the level of TAAb which specifically binds RAB7L1 is increased as compared to a corresponding healthy sample. In one embodiment, the level of TAAb which specifically binds RAB7L1 is decreased as compared to a corresponding healthy sample.

In one embodiment, the level of TAAb which specifically binds ACSBG1 is increased as compared to a corresponding healthy sample. In one embodiment, the level of TAAb which specifically binds ACSBG1 is decreased as compared to a corresponding healthy sample.

In one embodiment, the level of TAAb which specifically binds AFP is increased as compared to a corresponding healthy sample. In one embodiment, the level of TAAb which specifically binds AFP is decreased as compared to a corresponding healthy sample.

In one embodiment, the level of TAAb which specifically binds CSNK1A1L is increased as compared to a corresponding healthy sample. In one embodiment, the level of TAAb which specifically binds CSNK1A1L is decreased as compared to a corresponding healthy sample.

In one embodiment, the level of TAAb which specifically binds DHFR is increased as compared to a corresponding healthy sample. In one embodiment, the level of TAAb which specifically binds DHFR is decreased as compared to a corresponding healthy sample.

In one embodiment, the level of TAAb which specifically binds MBNL1 is increased as compared to a corresponding healthy sample. In one embodiment, the level of TAAb which specifically binds MBNL1 is decreased as compared to a corresponding healthy sample.

In one embodiment, the level of TAAb which specifically binds p53 is increased as compared to a corresponding healthy sample. In one embodiment, the level of TAAb which specifically binds p53 is decreased as compared to a corresponding healthy sample.

In one embodiment, the level of TAAb which specifically binds PRL is increased as compared to a corresponding healthy sample. In one embodiment, the level of TAAb which specifically binds PRL is decreased as compared to a corresponding healthy sample.

In one embodiment, the level of TAAb which specifically binds PSMC1 is increased as compared to a corresponding healthy sample. In one embodiment, the level of TAAb which specifically binds PSMC1 is decreased as compared to a corresponding healthy sample.

In one embodiment, the level of TAAb which specifically binds PTGFR is increased as compared to a corresponding healthy sample. In one embodiment, the level of TAAb which specifically binds PTGFR is decreased as compared to a corresponding healthy sample.

In one embodiment, the level of TAAb which specifically binds PTPRA is increased as compared to a corresponding healthy sample. In one embodiment, the level of TAAb which specifically binds PTPRA is decreased as compared to a corresponding healthy sample.

In one embodiment, the level of TAAb which specifically binds SCYL3 is increased as compared to a corresponding healthy sample. In one embodiment, the level of TAAb which specifically binds SCYL3 is decreased as compared to a corresponding healthy sample.

In still another aspect, the invention provides an array which may be used to detect the level of biomarker(s) and/or TAAb(s) of the present invention. In one embodiment the array includes immobilized biomolecules capable of selectively binding or hybridizing to a genomic sequence, transcript, or protein of one or more of CA125, CA15.3, CA19-9, HE4, Prolactin, APOA1, CLDN1, CLDN3, CLDN4, CLDN5, CLDN7, VEGF, LPA, AFP, LH, CEA and FSH.

In one embodiment the array includes immobilized biomolecules capable of selectively binding or hybridizing to one or more TAAbs which selectively bind one or more of RAB7L1, ACSBG1, AFP, CSNK1A1L, DHFR, MBNL1, p53, PRL, PSMC1, PTGFR, PTPRA, SCYL3 or variants thereof.

In various aspects the biomolecules include antibodies or oligonucleotides, such as nucleic acid primers or probes.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, for example, whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, e.g., Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

Another aspect of the present invention is that the assay can be provided in a kit which allows for more convenient laboratory-based biomarker analysis. The kits may include a plurality of components including reagents, supplies, written instructions, and/or software. The kits may have a plurality of embodiments including laboratory kits and mail-in kits. The kits can include secondary reagents. Secondary reagents may be antibodies, enzymes, labels, or chemicals and may enable a complete biomarker panel assay.

Exemplary kits can include at least one means for detection of one or more of the disclosed panel constituents (such as, at least two, at least three, at least four, or at least five detection means). In some examples, such kits can further include at least one means for detection of one or more (e.g., one to three) housekeeping genes or proteins. Detection means can include, without limitation, a nucleic acid probe specific for a genomic sequence including a disclosed gene, a nucleic acid probe specific for a transcript (e.g., mRNA) encoded by a disclosed gene, a pair of primers for specific amplification of a disclose gene (e.g., genomic sequence or cDNA sequence of such gene), an antibody or antibody fragment specific for a protein encoded by a disclosed gene.

In some kit embodiments, the primary detection means (e.g., nucleic acid probe, nucleic acid primer, or antibody) can be directly labeled, e.g., with a fluorophore, chromophore, or enzyme capable of producing a detectable product (such as alkaline phosphates, horseradish peroxidase and others commonly known in the art). Other kit embodiments will include secondary detection means; such as secondary antibodies (e.g., goat anti-rabbit antibodies, rabbit anti-mouse antibodies, anti-hapten antibodies) or non-antibody hapten-binding molecules (e.g., avidin or streptavidin). In some such instances, the secondary detection means will be directly labeled with a detectable moiety. In other instances, the secondary (or higher order) antibody will be conjugated to a hapten (such as biotin, DNP, and/or FITC), which is detectable by a detectably labeled cognate hapten binding molecule (e.g., streptavidin (SA) horseradish peroxidase, SA alkaline phosphatase, and/or SA QDot™). Some kit embodiments may include colorimetric reagents (e.g., DAB, and/or AEC) in suitable containers to be used in concert with primary or secondary (or higher order) detection means (e.g., antibodies) that are labeled with enzymes for the development of such colorimetric reagents.

In some embodiments, a kit includes positive or negative control samples, such as a cell line or tissue known to express or not express a particular biomarker.

In some embodiments, a kit includes instructional materials disclosing, for example, means of use of a probe or antibody that specifically binds a disclosed gene or its expression product (e.g., microRNA, mRNA or protein), or means of use for a particular primer or probe. The instructional materials may be written, in an electronic form (e.g., computer diskette or compact disk) or may be visual (e.g., video files). The kits may also include additional components to facilitate the particular application for which the kit is designed. Thus, for example, the kit can include buffers and other reagents routinely used for the practice of a particular disclosed method. Such kits and appropriate contents are well known to those of skill in the art.

Certain kit embodiments can include a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes a one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still other kit embodiments may include disposal means for discarding used or no longer needed items (such as subject samples). Such disposal means can include, without limitation, containers that are capable of containing leakage from discarded materials, such as plastic, metal or other impermeable bags, boxes or containers.

The kits can further include software. Software may include a training video that may provide additional support including demonstration of biomarker assays, examples of results, or educational materials for performing biomarker assays according to the invention.

In a related embodiment, the array of the present invention may be included in the kit of the present invention optionally along with reagents for performing an array based assay.

Polynucleotides or nucleic acid sequences of the present invention, such as oligonucleotides, primers, probes, and the like may be of any suitable length. For example, one of skill in the art would understand what lengths are suitable for nucleic acid sequences to be used in an array or kit of the invention. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Detection of Ovarian Cancer

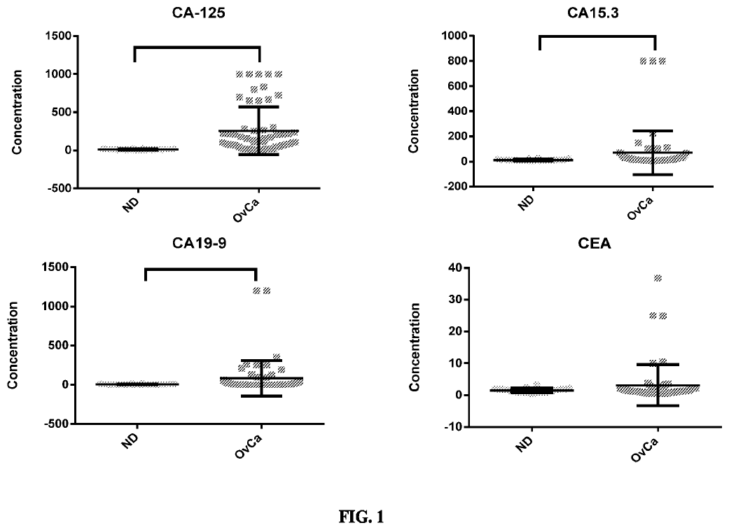

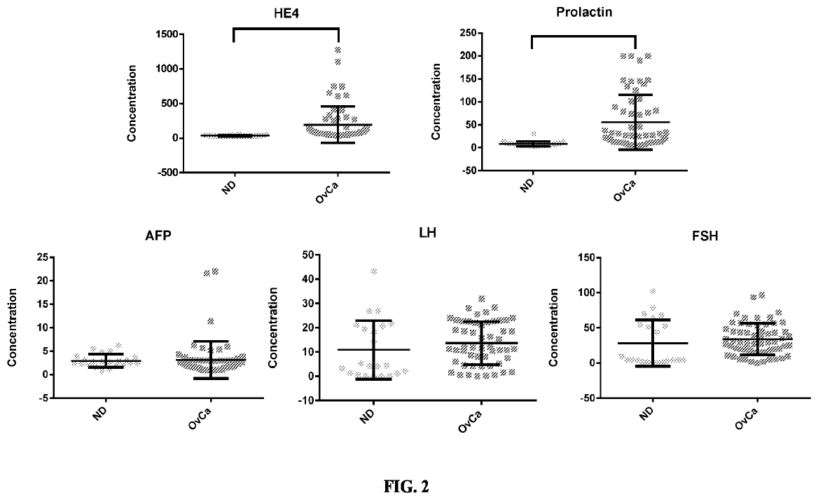

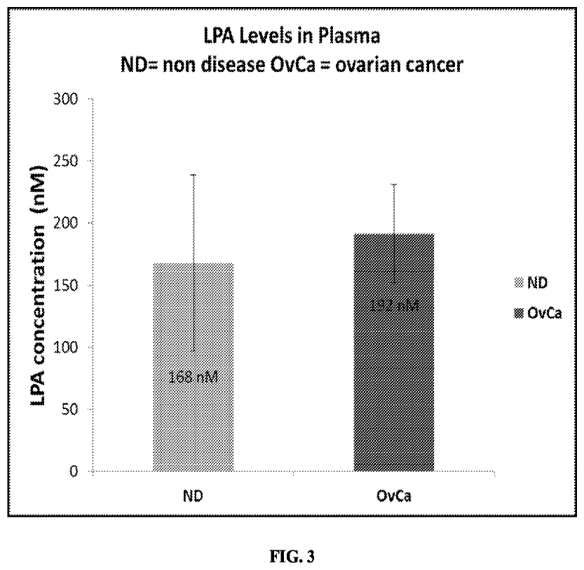

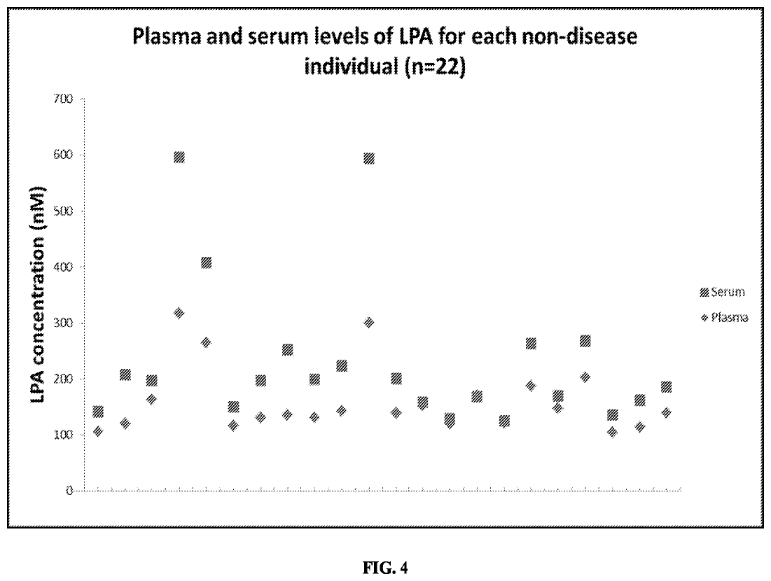

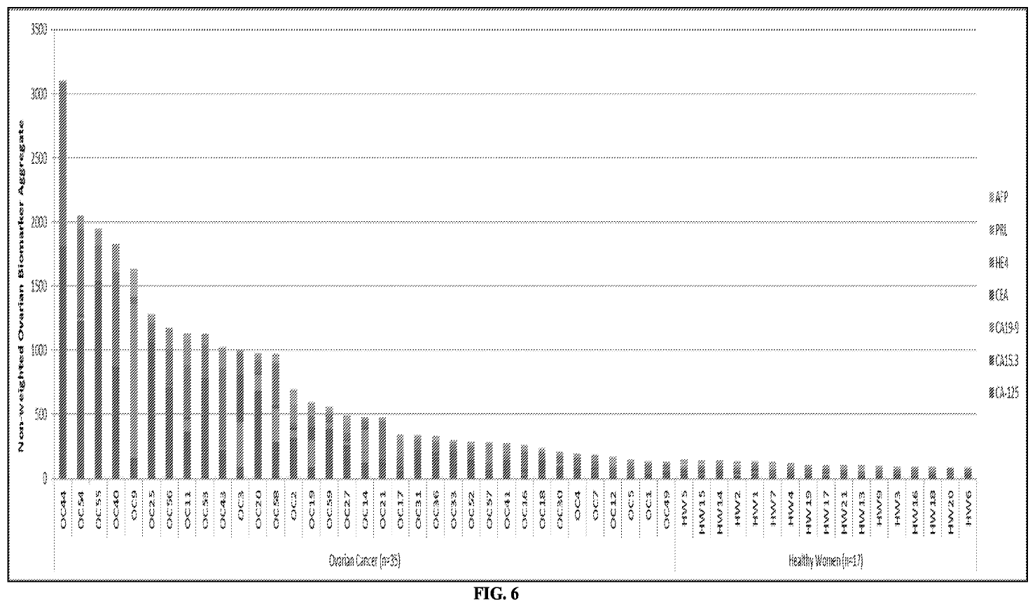

All serum protein concentrations (CA125, CA15.3, CA19-9, HE4, Prolactin, LPA, AFP, LH, CEA and FSH) were determined using the Abbott Architect Plus® (Abbott Laboratories). Results are shown in FIGS. 1-4 and 6. All assays were performed according to manufacturer's specifications. Briefly, microparticles coated with target-specific monoclonal antibodies are added to serum and allowed to bind. The microparticles are washed and a conjugated secondary antibody is added. Solutions of 1.32% hydrogen peroxide and 0.35N sodium hydroxide are added to initiate and stop, respectively, the chemiluminescent reaction. The signal is measured as relative light units (RLU) and converted to concentration values based on manufacturer calibration standards. Sample values must fall within the calibration curve to be counted as a valid sample.

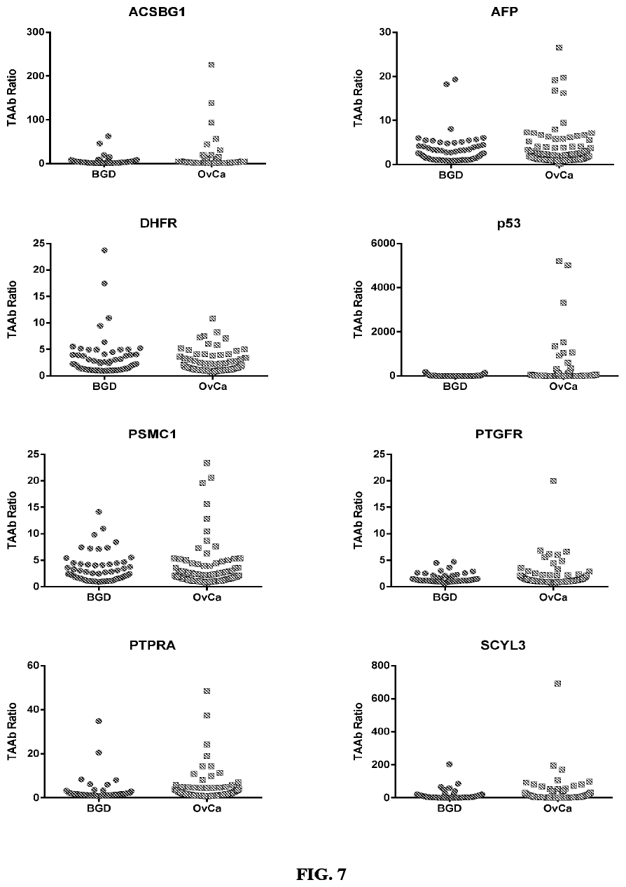

Detection of TAAbs which specifically bind RAB7L1, ACSBG1, AFP, CSNK1A1L, DHFR, MBNL1, p53, PRL, PSMC1, PTGFR, PTPRA and SCYL3 was performed by ELISA (TAAb Indirect ELISA). Recombinant full-length proteins (manufactured by Origene Technologies and Abnova Corporation) were coated onto standard blank 384-well uncoated plates (MSD) and incubated overnight at 4° C. Assay workflow followed a standard indirect ELISA protocol. In brief, samples and plates are blocked using a 1:10 KPL Milk solution (KPL Laboratories) with the samples being diluted at a 1:10 ratio. Diluted samples were added to the plate in duplicate wells. Anti-human CH2 (Pierce) was diluted in 1:10 KPL Milk and added to each well, followed by diluted SULFO tagged anti-mouse antibody (MSD). All plates were analyzed using a MSD S-600™ imager. To be deemed valid, a sample must have a duplicate coefficient of variation (CV) value below 20%. Any samples with duplicate measurements outside of this range were re-run to ensure a valid sample. Results of the detection are shown in FIG. 7.

Although the invention has been described with reference to the above example, it will be understood that modifications and variations are encompassed within the spirit and scope of the invention. Accordingly, the invention is limited only by the following claims.

What is claimed is:

1. A method for measuring the level of a protein biomarker and an autoantibody in a sample from a subject having or at risk of having ovarian cancer comprising:
a) obtaining a biological sample from the subject; and
b) measuring a level of at least one protein biomarker and at least one autoantibody, wherein the at least one protein biomarker is selected from CA125, CA15.3, CA19-9, HE4, Prolactin, APOA1, CLDN1, CLDN3, CLDN4, CLDN5, CLDN7, VEGF, LPA, AFP, LH, CEA, FSH, and any combination thereof, and wherein the at least one autoantibody specifically binds RAB7L1, ACSBG1, AFP, CSNK1A1L, DHFR, MBNL1, p53, PRL, PSMC1, PTGFR, PTPRA, SCYL3, or any combination thereof.

2. The method of claim 1, wherein the sample is a bodily fluid such as ascites, serum, plasma, feces, lymph, cerebrospinal fluid, nipple aspirate, or urine.

3. The method of claim 1, wherein the at least one protein biomarker comprises HE4, CA15.3, CA125, CA19-9, Prolactin, or any combination thereof.

4. The method of claim 1, wherein the at least one autoantibody specifically binds AFP, CSNK1A1L, p53, PRL, PTGFR, PTPRA, or any combination thereof.

5. The method of claim 1, wherein the at least one autoantibody specifically binds AFP, CSNK1A1L, p53, PRL, PTGFR, PTPRA, or any combination thereof, and the at least one protein biomarker comprises HE4, CA15.3, CA125, CA19-9, Prolactin, or any combination thereof.

6. The method of claim 1, wherein the at least one autoantibody specifically binds AFP, CSNK1A1L, p53, PRL, PTGFR and PTPRA, and the at least one protein biomarker comprises HE4, CA15.3, CA125, CM 9-9 and Prolactin.

7. The method of claim 1, wherein the at least one autoantibody specifically binds RAB7L1, ACSBG1, AFP, CSNK1A1L, DHFR, MBNL1, p53, PRL, PSMC1, PTGFR, PTPRA and SCYL3.

8. The method of claim 1, wherein the at least one protein biomarker comprises CA125, CA15.3, CA19-9, HE4, Prolactin, APOA1, CLDN1, CLDN3, CLDN4, CLDN5, CLDN7, VEGF, LPA, AFP, LH, CEA and FSH.

9. The method of claim 1, wherein the at least one protein biomarker comprises CA125, CA15.3, CA19-9, HE4, Prolactin, LPA, AFP, LH, CEA and FSH.

10. The method of claim 1, wherein the at least one autoantibody specifically binds RAB7L1, ACSBG1, AFP, CSNK1A1L, DHFR, MBNL1, p53, PRL, PSMC1, PTGFR, PTPRA and SCYL3, and the at least one protein biomarker comprises CA125, CA15.3, CA19-9, HE4, Prolactin, APOA1, CLDN1, CLDN3, CLDN4, CLDN5, CLDN7, VEGF, LPA, AFP, LH, CEA and FSH.

11. The method of claim 1, wherein the at least one autoantibody specifically binds RAB7L1, ACSBG1, AFP, CSNK1A1L, DHFR, MBNL1, p53, PRL, PSMC1, PTGFR, PTPRA and SCYL3, and the at least one protein biomarker comprises CA125, CA15.3, CA19-9, HE4, Prolactin, LPA, AFP, LH, CEA and FSH.

12. The method of claim 1, wherein the method further comprises histological analysis of a biopsy tissue.

13. The method of claim 1, wherein the method further comprises image analysis.

14. The method of claim 1, wherein the level of the at least one protein biomarker is determined via protein array analysis.

15. The method of claim 1, wherein the subject is a mammal.

16. The method of claim 1, wherein the mammal is a human.

17. The method of claim 1, further comprising administering a therapeutic agent to the subject.

18. The method of claim 1, further comprising prescribing the subject a therapeutic regime.

19. The method of claim 1, wherein (b) comprises measuring an expression product of the at least one protein biomarker or the at least one autoantibody.

20. The method of claim 19, wherein the expression product is protein, microRNA or mRNA.

21. A kit for detecting ovarian cancer in a subject, comprising means for detecting in a biological sample at least one protein biomarker and at least one autoantibody, the at least one protein biomarker being a genomic sequence, transcript, or protein of CA125, CA15.3, CA19-9, HE4, Prolactin, APOA1, CLDN1, CLDN3, CLDN4, CLDN5, CLDN7, VEGF, LPA, AFP, LH, CEA, FSH, or any combination thereof.

22. The kit of claim 21, wherein the at least one protein biomarker is a genomic sequence, transcript, or protein of CA125, CA15.3, CA19-9, HE4, Prolactin, LPA, AFP, LH, CEA and FSH.

23. The kit of claim 21, wherein the at least one autoantibody specifically binds RAB7L1, ACSBG1, AFP, CSNK1A1L, DHFR, MBNL1, p53, PRL, PSMC1, PTGFR, PTPRA, SCYL3, or any combination thereof.

24. The kit of claim 21, further comprising a container suitable for containing the means and the biological sample.

25. The kit of claim 21, wherein the kit comprises a nucleic acid probe specific for the biomarker or autoantibody.

26. The kit of claim 21, wherein the kit comprises a pair of primers for specific amplification of a transcript of the biomarker or binding partner of the autoantibody.

27. The kit of claim 21, wherein the kit comprises an antibody specific for the biomarker or autoantibody.

28. The kit of claim 21, wherein the kit comprises reagents for detecting protein, microRNA or mRNA.

29. An array comprising a plurality of probes for specifically binding a biomarker and an autoantibody, wherein the biomarker is at least one or more of CA125, CA15.3, CA19-9, HE4, Prolactin, APOA1, CLDN1, CLDN3, CLDN4, CLDN5, CLDN7, VEGF, LPA, AFP, LH, CEA and FSH.

30. The array of claim 29, wherein the at least one autoantibody specifically binds RAB7L1, ACSBG1, AFP, CSNK1A1L, DHFR, MBNL1, p53, PRL, PSMC1, PTGFR, PTPRA, SCYL3, or any combination thereof.

31. The array of claim 29, wherein the plurality of probes are oligonucleotides.

32. The array of claim 29, wherein the plurality of probes are polypeptides.

33. The array of claim 32, wherein the plurality of probes are antibodies.

34. A panel for detecting ovarian cancer in a subject, the panel comprising:
(a) reagents for detecting at least one protein biomarker, the at least one protein biomarker being a genomic sequence, transcript, protein, or protein fragment selected from CA125, CA15.3, CA19-9, HE4, Prolactin, APOA1, CLDN1, CLDN3, CLDN4, CLDN5, CLDN7, VEGF, LPA, AFP, LH, CEA, FSH, and any combination thereof; and
(b) reagents for detecting one or more autoantibodies, wherein the one or more autoantibodies specifically bind RAB7L1, ACSBG1, AFP, CSNK1A1L, DHFR, MBNL1, p53, PRL, PSMC1, PTGFR, PTPRA, SCYL3, or any combination thereof.

35. A method for detecting ovarian cancer, or risk thereof, in a subject, comprising:
a) obtaining a biological sample from the subject; and
b) measuring a level of at least one protein biomarker and at least one autoantibody, both as compared with a healthy subject's sample; wherein a level of antibody and biomarker greater than that found in the healthy sample, is indicative of a subject having or at risk of having ovarian cancer, wherein the at least one protein biomarker is selected from CA125, CA15.3, CA19-9, HE4, Prolactin, APOA1, CLDN1, CLDN3, CLDN4, CLDN5, CLDN7, VEGF, LPA, AFP, LH, CEA, FSH, and any combination thereof, and wherein the at least one autoantibody specifically binds RAB7L1, ACSBG1, AFP, CSNK1A1L, DHFR, MBNL1, p53, PRL, PSMC1, PTGFR, PTPRA, SCYL3, or any combination thereof.

36. The method of claim 35, wherein the sample is a bodily fluid such as ascites, serum, plasma, feces, lymph, cerebrospinal fluid, nipple aspirate, or urine.

37. The method of claim 35, wherein the at least one protein biomarker comprises HE4, CA15.3, CA125, CA19-9, Prolactin, or any combination thereof.

38. The method of claim 35, wherein the at least one autoantibody specifically binds AFP, CSNK1A1L, p53, PRL, PTGFR, PTPRA, or any combination thereof.

39. The method of claim 35, wherein the at least one autoantibody specifically binds AFP, CSNK1A1L, p53, PRL, PTGFR, PTPRA, or any combination thereof, and the at least one protein biomarker comprises HE4, CA15.3, CA125, CA19-9, Prolactin, or any combination thereof.

40. The method of claim 35, wherein the at least one autoantibody specifically binds AFP, CSNK1A1L, p53, PRL, PTGFR and PTPRA, and the at least one protein biomarker comprises HE4, CA15.3, CA125, CA19-9 and Prolactin.

41. The method of claim 35, wherein the at least one autoantibody specifically binds RAB7L1, ACSBG1, AFP, CSNK1A1L, DHFR, MBNL1, p53, PRL, PSMC1, PTGFR, PTPRA and SCYL3.

42. The method of claim 35, wherein the at least one protein biomarker comprises CA125, CA15.3, CA19-9, HE4, Prolactin, APOA1, CLDN1, CLDN3, CLDN4, CLDN5, CLDN7, VEGF, LPA, AFP, LH, CEA and FSH.

43. The method of claim 35, wherein the at least one protein biomarker comprises CA125, CA15.3, CA19-9, HE4, Prolactin, LPA, AFP, LH, CEA and FSH.

44. The method of claim 35, wherein the at least one autoantibody specifically binds RAB7L1, ACSBG1, AFP, CSNK1A1L, DHFR, MBNL1, p53, PRL, PSMC1, PTGFR, PTPRA and SCYL3, and the at least one protein biomarker comprises CA125, CA15.3, CA19-9, HE4, Prolactin, APOA1, CLDN1, CLDN3, CLDN4, CLDN5, CLDN7, VEGF, LPA, AFP, LH, CEA and FSH.

45. The method of claim 35, wherein the at least one autoantibody specifically binds RAB7L1, ACSBG1, AFP, CSNK1A1L, DHFR, MBNL1, p53, PRL, PSMC1, PTGFR, PTPRA and SCYL3, and the at least one protein biomarker comprises CA125, CA15.3, CA19-9, HE4, Prolactin, LPA, AFP, LH, CEA and FSH.

46. The method of claim 35, wherein the method further comprises histological analysis of a biopsy tissue.

47. The method of claim 35, wherein the method further comprises image analysis.
